(19) United States
(12) Patent Application Publication
(10) Pub. No.: US 2009/0148877 A1
(43) Pub. Date: Jun. 11, 2009

(54) ENZYMATIC METHODS FOR MEASURING PLASMA AND TISSUE SPHINGOMYLELIN AND PHOSPHATIDYLCHOLINE

(75) Inventors: Xian-Cheng Jiang, Fort Lee, NJ (US); Mohammad Reza Hojjati, Brooklyn, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: THE RESEARCH FOUNDATION OF STATE UNIVERSITY OF NEW YORK, Albany, NY (US)

(21) Appl. No.: 12/097,380

(22) PCT Filed: Dec. 13, 2006
(86) PCT No.: PCT/US06/47652

§ 371 Date: Oct. 23, 2008

Related U.S. Application Data
(60) Provisional application No. 60/750,629, filed on Dec. 15, 2005.

(51) Int. Cl. C12Q 1/42

(52) U.S. Cl. 435/21

(57) ABSTRACT

A method for measuring sphingomyelin and phosphatidylcholine comprising incubating sphingomyelin and phosphatidylcholine with bacterial sphingomyelinase and bacterial phospholipase D, alkaline phosphatase, choline oxidase, peroxidase, N-Ethyl-N-(2-hydroxy-3-sulfopropyl)-3,5-dimethoxyaniline, and 4-aminoantipyrine, preferably for about 45 minutes. A blue dye is generated.

### BACKGROUND OF THE INVENTION

Besides cholesterol and triglycerides, lipoproteins also contain phospholipids, among them phosphatidylcholine (PC) and sphingomyelin (SM) are two major ones, the former comprising about 70% and laTter about 20% of total phospholipids. In a human case-control study, it was indicated that both plasma SM and SM/PC ratio are independent risk factors for coronary heart disease.

It has been known for some time that SM accumulates in atheromas in human and animal models. Low density lipoprotein (LDL) extracted from human atherosclerotic lesions is much richer in SM than LDL from plasma. Plasma SM levels in apoE knockout (apoE KO) mice are 4-fold higher than in wild type mice, and this may partly explain the increased atherosclerosis in these animals. The SM/PC ratio was 5-fold higher in VLDL from hypercholesterolemic rabbits.

Recently, it has been demonstrated that administration of myriocin (an inhibitor of SM synthesis) into apoE KO mice dramatically decrease SM, increases PC and thus decreases SM/PC ratio in the plasma, and significantly decreased the atherosclerotic lesion area. These data suggest that SM might play a promoting role, while PC might play a preventive role, in the development of atherosclerosis. Their measurements might provide new insights into atherogenesis in humans and in various mouse models as well.

Although the importance of both phospholipids is very obvious, there are no simple, rapid, sensitive and high-throughput methods for their measurements. Classically, plasma SM and PC were measured by lipid extraction, thin layer chromatograph, and phosphate determination on separated SM or PC spots. This method is time-consuming and not sensitive,

Accordingly, there is a need for new methods of measuring SM and PC.

### SUMMARY OF THE INVENTION

The invention relates to a method for measuring plasma and tissue sphingomylelin and phosphatidylcholine comprising 1) catalyzing the hydrolysis of sphingomylelin to phosphorylcholine and n-acylsphingosine with bacterial SMase; 2) generating choline from phosphorylcholine produced from step 1) with alkaline phosphatase; 3) generating hydrogen peroxide by adding choline oxidase; and 4) adding hydrogen peroxide and with DAOS N-Ethyl-N-(2-hydroxy-3-sulfopropyl)-3,5-dimethoxyaniline, sodium salt), 4-aminoantipyrine and peroxidase, to generate a blue to purple dye, preferably with an optimal absorption at 595 nm.

In another embodiment, the method comprises 1) catalyzing the hydrolysis of phosphatidlycholine to choline and phosphatidic acid with bacterial phospholipase D; 2) generating hydrogen peroxide by adding choline oxidase; and 3) adding hydrogen peroxide and with DAOS (N-Ethyl-N-(2-hydroxy-3-sulfopropyl)-3,5-dimethoxyaniline, sodium salt), 4-aminoantipyrine and peroxidase, to generate a blue to purple dye, preferably with an optimal absorption at 595 nm.

In another embodiment, both phosphatidlycholine and sphingomylelin are measured concurrently by combining the two methods described above.

### DETAILED DESCRIPTION OF THE INVENTION DISCUSSION

Classically, SM and PC were measured by four steps: 1) lipid extraction; 2) thin layer chromatograph (TLC); 3) SM and PC extraction from corresponding spots on the TLC plate, and 4) quantification of phosphate in each extraction. The whole procedure is time-consuming and not sensitive. Although there is a simple method for measuring total choline-containing phospholipids (PC+SM) (Wako Pure Chemical), there is no corresponding method for direct SM and PC measurements. The invention relates to two rapid, specific and sensitive assays for plasma SM and PC measurements.

The invention relates to two rapid, specific and sensitive enzymatic measurements for both Sphingomyelin (SM) and phosphatidylcholine (PC). (SM) and (PC) are two major phospholipids on plasma lipoproteins. Their concentration is classically measured by lipid extraction, thin layer chromatograph, and phosphate determination on separated SM or PC spots.

In the inventive method, plasma is incubated with bacterial sphingomyelinase (for SM measurement) or bacterial phospholipase V (for PC measurement), alkaline phosphatase, choline oxidase, peroxidase, N-Ethyl-N-(2-hydroxy-3-sulfopropyl )-3,5-dimethoxyaniline, and 4-aminoantipyrine, preferably for about 45 minutes. A blue dye, with an optimal absorption at 595 nm, is generated.

PC levels do not influence SM measurement or vice versa. The linear range for the SM measurement is about 0.5 to about 5 μg and for PC was about 2.5 to about 20 μg. The inter-assay coefficient of variation of the assay was about 1.7±0.05% for SM and 3.1±0.13% for PC. These two methods are amenable to automation and can be adopted for large-scale, high-throughput assays.

Using SMase and phospholipaes D render the specificity of our assays. However, not all the commercial available enzymes are usefule. Some of phospholipase D might contaminate with SMase activity or vice versa, Preferably phospholipase D from BIOMOL International is used in the inventive method or assay.

The terms “assay” and “method” have the same meaning herein and are used interchangeably herein.

SMase available in Sigma-Aldrich can be used in the inventive method, preferably S-8889. All alkaline phosphatase, choline oxidase, and peroxidase available in Sigma-Aldrich can be used on in all of the methods of the invention.

In the last step of the inventive method, i.e. converting H₂O₂ into a readable compound, some reagents can be chosen. For instance, phenol can be used to generate a red quinine pigment, with an optimal absorption at 505 nm and TOOS (3-(N-ethyl-3-methylanilino)-2-hydroxypropanesulfonic acid) can be used to generate a purple pigment, with an optimal absorption at 550 nm. However, hemolytic plasma could significantly influence the absorption at both wave lengths. Utilizing DAOS could sufficiently avoid the effect of hemolysis (FIG. 4).

The novel methods for plasma SM and PC measurement described herein are simple, rapid, specific, sensitive and has high throughput. They are suitable for larger scale clinical samples measurements or drug screening and may be adaptive for tissue SM and PC measurements.

With the aim to better illustrate the present invention, without posing any limitation to it, the following examples are now given.

### MATERIALS AND METHODS

Reagents: SMase, alkaline phosphatase, choline oxidase, peroxidase and 4-aminoantipyrine as well as standard SM and standard PC were purchased from Sigma-Aldrich. Phospholipase D was purchased from BIOMOL International. DAOS (N-ethyl-N-(2-hydroxy-3-sulfopropyl)-3,5-dimethoxyaniline, sodium salt) was purchased from Dojindo Molecular Technologies, Inc.

SM measurement: There were four steps for enzymatic measurement of plasma SM levels (FIG. 1A): 1) Bacterial SMase hydrolyzed SM to phosphorylcholine and n-acylsphingosine; 2) alkaline phosphatase generated choline from phosphorylcholine; 3) choline was used to generate hydrogen peroxide in a reaction catalyzed by choline oxidase; and 4) hydrogen peroxide was used together with DAOS (N-Ethyl-N-(2-hydroxy-3-sulfopropyl)-3,5-dimethoxyaniline, sodium salt), 4-aminoantipyrine and peroxidase, as a catalyst, to generate a blue to purple dye, with an optimal absorption at 595 nm. The reaction buffer was Tris-HCl 0.05 M with calcium chloride 5 mg/dl, pH 8. Enzymes concentrations in a 50 ml reaction buffer were as follows: SMase 25U, alkaline phosphatase 500U, choline oxidase 25U, and peroxidase 1000U. DAOS concentration was 0.73 mM and 4-aminoantipyrine concentration was 0.73 mM. Five μl of plasma were added to 100 μl reaction buffer plus enzymes and after 45 minutes incubation at 37° C., the absorption was measured at 595 nm on a spectrophotometric plate reader. Standard SM solution (50 mg/dl) preparation: 5 mg of SM was dissolved in 10 ml 2% Triton X-100 ethanol solution.

PC measurement: There were three steps for enzymatic measurement of plasma PC levels (FIG. 2B): 1) Bacterial phospholipase D (specific for PC, no reaction with SM) hydrolyzed PC to choline and phosphatidic acid; 2) choline was used to generate hydrogen peroxide in a reaction catalyzed by choline oxidase; 3) hydrogen peroxide was used together with DAOS (N-Ethyl-N-(2-hydroxy-3-sulfopropyl)-3,5-dimethoxyaniline, sodium salt), 4-aminoantipyrine and Peroxidase, as a catalyst, to generate a blue to purple dye, with an optimal absorption at 595 nm. The reaction buffer was Tris-HCL 0.05 M with calcium chloride 5 mg/dl, pH 7. Enzymes concentrations in a 50 ml reaction buffer were as follows: Phospholipase D 6000U (added at the time of measurement), Choline oxidase 25U, Peroxidase 1000U. DAOS concentration was 0.73 mM and 4-aminoantipyrine concentration was 0.73 mM. Five μl of plasma were added to 100 μl reaction buffer plus enzymes and after 45 minutes incubation at 37° C., the absorption was measured at 595 nm, Standard SM solution (100 mg/dl) preparation: 10 mg of PC was dissolved in 10 ml 2% Triton X-100 ethanol solution.

Total phospholipids measurement: The total choline-containing phospholipids (PC+SM) in plasma was measured by an enzymatic method (Wako Pure Chemical)

### RESULTS

Enzymatic measurement of plasma SM or PC levels were carried out by using novel 4- or 3-step procedure (FIG. 1). As indicated in FIG. 2, the linear range for the SM measurement was 0.5 to 5 μg and for PC was 2.5 to 20 μg (FIG. 2). SM and PC concentration were measured in different amount of pooled plasma and found that the linear range for both assays was 2.5 μl to 10 μl (FIG. 3).

Since both methods are very similar except the first step (FIG. 1), it is likely that both measurements would interfere with each other. To investigate the specificity for the SM method, standard PC as a substrate or vice versa, and there was no crossing measurement in both methods (FIGS. 4A and 4B).

Hemolysis is always occurred during the blood collecting. Since plasma SM concentration is significantly lower than cholesterol and PC, the hemolytic plasma may significantly interfere with the absorption reading of the assay. To investigate this effect, 10 μl of low, medium and high hemolytic plasma samples was utilized and incubated with SM assay solution but without SMase at 37° C. for 45 min, and their absorption was measured at 595 nm. It was found that that hemolysis did not interfere with SM assay (FIG. 5).

Reproducibility of methods: SM and PC were measured in one sample 20 times. The interassay coefficient of variation of the SM assay was 1.7±0.05% and PC assay was 3.1±0.13%.

To validate the novel SM and PC assays, total choline-containing phosphlipid (SM+PC) levels in mouse plasma was measured using a commercial available kit (Wako Pure Chemical), and these results were compared with the results obtained by adding SM and PC concentrations measured by the inventive methods. It was found that the two approaches were correlated well (r=0.91, n=7) (FIG. 6). Moreover, mouse plasma SM concentration was 38±10 mg/dl and PC was 150±21 mg/dl, PC/SM ratio was 3.9 (Table 1). All these results were comparable with those obtained by the classical methods (7).

TABLE 1
Comparison of the new methods for plasma SM and PC measurements
with a commercial available kit which measuring total choline-containing
phospholipids (PC + SM).

| | PC* | | | |
|---|---|---|---|---|
| SM* (mg/dl) | (mg/dl) | PC + SM (mg/dl) | PC/SM | PL** (mg/dL) |
| 38 ± 10 | 150 ± 31 | 189 ± 29 | 3.9 ± 1.0 | 201 ± 37 |

*Measured by the new methods.
**Measured by a commercial Kit (Wako Pure Chemical).
SM, sphingomyelin;
PC, phosphatidylcholine.
Values are mean ± SD, n = 17.

Modification of the SM measurement method: In order to increase the sensitivity of the SM measurement method as well as to avoid the effect of hemolysis, DAOS was instead of phenol in the last step of the reaction with the highest absorption at 595 nm. This change not only increases the sensitivity of the method (less than 10 mg/dl of SM can be detected) but also avoids the effect of hemolysis.

Standard curve for the SM and PC measurements: Standard curve with the standard SM (0.35 to 3.5 μg) was linear for the SM measurement method. Standard curve with the standard PC (6 to 24 μg) was linear for the PC measurement method. The linear range of plasma SM in the assay was between 10 and 120 mg/dl. The linear range of plasma PC in the assay was between 10 and 250 mg/dl.

Reproducibility of Methods: Using the new methods, SM and PC was measured in one sample 20 times. The interassay coefficient of variation of the SM assay was 1.7±0.05%. The interassay coefficient of variation of the PC assay was 3.1±0.13%.

### BRIEF DESCRIPTION OF THE FIGURES

FIG. 1. Strategy for SM and PC measurements. A. SMase catalyzes hydrolysis of SM to phosphorylcholine, alkaline phosphatase catalyzes in the second step in which P-choline produces choline. Oxidation of choline is the next step that catalyzes by choline oxidase. This reaction produces two hydrogen peroxides. The last step catalyzes by peroxidase, which produces a purple to blue dye that can be measured. B. Phospholipase D catalyzes the hydrolysis of PC to choline and phosphatidic acid. The rest of the reactions are similar to the SM measurement.

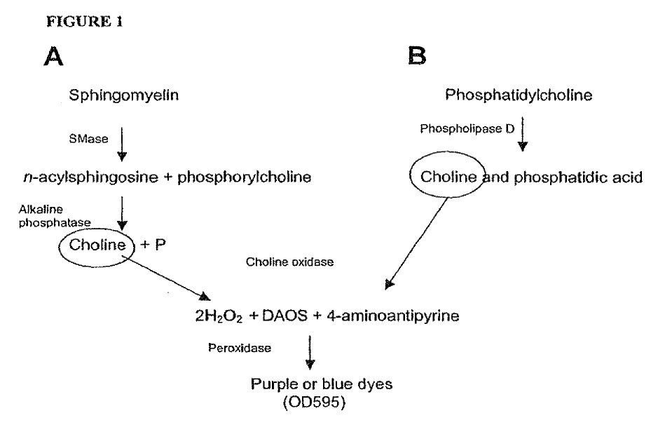

FIG. 2. Standard curve for the SM and PC measurements. Different amount of SM or PC standard solution supplemented with saline to 20 μl was incubated with 100 μl of reaction buffer at 37° C. for 45 min, the absorption was measured at 595 nm. A. Standard curve for SM. B. Standard curve for PC.

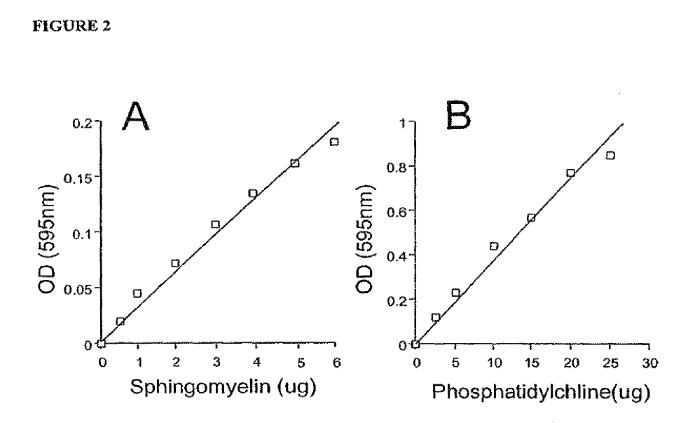

FIG. 3. Linear range of plasma SM and PC measurements. Pooled mouse plasma was used. Different amount of the plasma supplemented with saline to 20 μl was incubated with 100 μl of reaction buffer at 37° C. for 45 min, the absorption was measured at 595 nm. A. Plasma linear range for SM measurement. B. Plasma linear range for PC measurement.

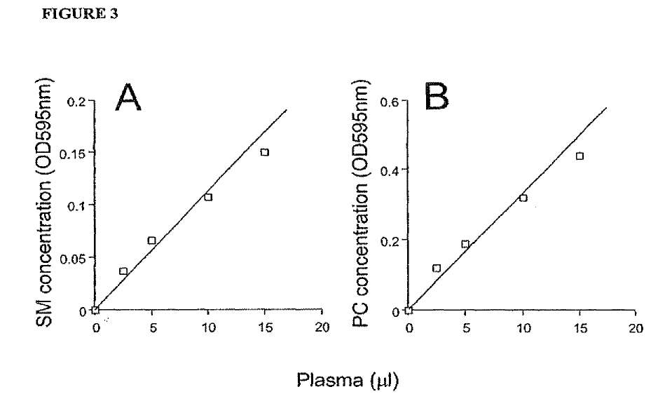

FIG. 4. Specificity of the SM and PC measurements. A. Different concentration of PC was used in the SM method; B. Different concentration of SM was used in the PC method.

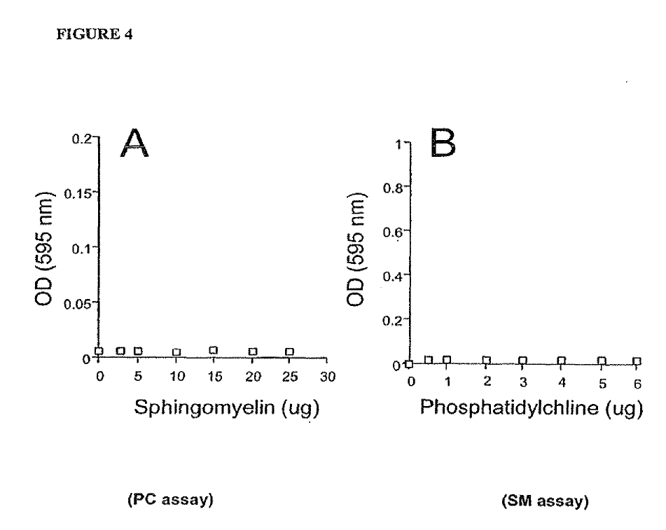

FIG. 5. The effect of hemolysis on OD reading at 595 nm. Ten μl of low, medium and high hemolytic plasma samples are incubated with 100 μl of SM assay solution but without SMase at 37° C. for 45 min, and their absorption was measured at 595 nm. BKG: Background; LOW: Low hemolysis; MED: Medium hemolysis; HIGH: High hemolysis.

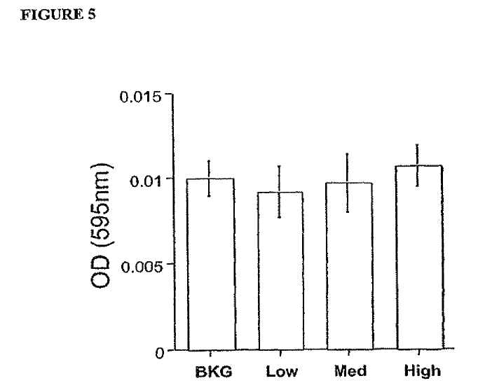

FIG. 6. Comparison of the new methods for plasma SM and PC measurements with a commercial available kit (Wako) which measuring total choline-containing phospholipid. r=0.91, n=17.

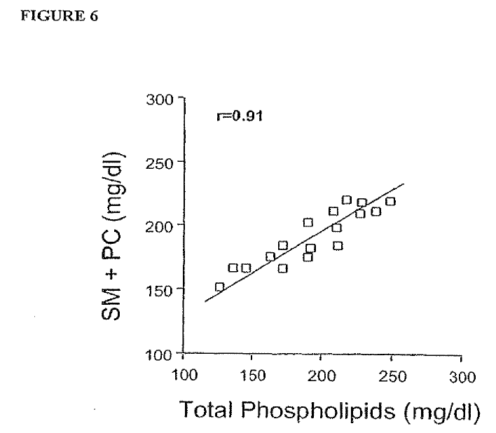

What is claimed is:

1. A method for measuring plasma and tissue sphingomylelin and phosphatidylcholine comprising 1) catalyzing the hydrolysis of sphingomylelin to phosphorylcholine and n-acylsphingosine with bacterial SMase; 2) generating choline from phosphorylcholine produced from step 1) with alkaline phosphatase; 3) generating hydrogen peroxide by adding choline oxidase; and 4) adding hydrogen peroxide and with DAOS (N-Ethyl-N-(2-hydroxy-3-sulfopropyl)-3,5-dimethoxyaniline, sodium salt), 4-aminoantipyrine and peroxidase, to generate a blue to purple dye.

2. A method for measuring plasma and tissue sphingomylelin and phosphatidylcholine comprising 1) catalyzing the hydrolysis of phosphatidlycholine to choline and phosphatidic acid with bacterial phospholipase D; 2) generating hydrogen peroxide by adding choline oxidase; and 3) adding hydrogen peroxide and with DAOS (N-Ethyl-N-(2-hydroxy-3-sulfopropyl)-3,5-dimethoxyaniline, sodium salt), 4-aminoantipyrine and peroxidase, to generate a blue to purple dye.

3. The method according to claim 1, further comprising catalyzing the hydrolysis of phosphatidlycholine to choline and phosphatidic acid with bacterial phospholipase D in step 1.
